(19) United States
(12) Patent Application Publication
(10) Pub. No.: US 2008/0027543 A1
(43) Pub. Date: Jan. 31, 2008

(54) Prosthesis and method for replacing degenerative vertebral portions

(75) Inventors: Lukas Eisermann, San Diego, CA (US); Marc Peterman, Austin, TX (US); Randall N. Allard, Germantown, TN (US)

Correspondence Address: FITZPATRICK CELLA HARPER & SCINTO, 30 ROCKEFELLER PLAZA, NEW YORK, NY, 10112, US

(73) Assignee: SDGI HOLDINGS, INC., Wilmington, DE (US)

(21) Appl. No.: 11/476,134

(22) Filed: Jun. 28, 2006

(51) Int. Cl. A61F 2/44

(52) U.S. Cl. 623/17.11

(57) ABSTRACT

A posterior arch prosthesis according to the present invention includes a main body, two transverse protuberances extending transversely from either side of the main body, articulating surfaces formed on each of the transverse protuberances, and a posterior protuberance extending posteriorly from the main body. The posterior protuberance is attached to a first vertebra from which the natural posterior arch has been removed such that the articulating surfaces are arranged in articulating contact with surfaces on an adjacent, second vertebra. The posterior protuberance includes an attachment to which native structure may be reattached upon attachment of the prosthesis to the first vertebra.

### BACKGROUND OF THE INVENTION

1. Field of the Invention

This invention relates generally to a prosthesis for replacing one or more portions of a vertebra. More specifically, this invention relates to a device for replacement of a posterior spinal arch and one or more vertebral posterior facets.

2. Description of the Related Art

As is known in the art, in the human anatomy, the spine is a generally flexible column that supports compressive and tensile loads, allows for bending, and provides a place of attachment for bones (i.e., ribs), muscles, and ligaments. In general, the spine is divided into the cervical, thoracic, and lumbar spinal sections. FIG. 1 illustrates the lumbar spinal section 1 and a sacrum region 3 in a healthy human.

Each section of the spine consists of individual bones, called vertebrae. FIGS. 2A and 2B depict, respectively, a side view and a posterior view of two adjacent, healthy vertebrae 20a, 20b, and FIG. 2C depicts a top view of a vertebra. Each vertebrae 20a, 20b generally includes a vertebral body 22 and two pedicles 23 extending posteriorly from the vertebral body 22, each terminating at a lamina 24. The laminae 24 adjoin, creating a posterior arch 29 bridging the pedicles 23. As most clearly shown in FIG. 2C, the posterior of the vertebral body 22, the pedicles 23, and the posterior arch 29 define an opening, or vertebral foramen, in which the spinal column is housed. Each vertebrae also includes two superior articular processes 25, two transverse processes 26, two inferior articular processes 28, and a spinous process 30 extending outwardly in various directions. Among other functions, the processes provide attachment areas for tendons and ligaments.

Vertebrae are generally aligned one on top of the other to form the spine, as shown in FIGS. 2A and 2B. In particular, the vertebral body 22 of a superior vertebra 20a generally aligns with the vertebral body 22 of an inferior vertebra 20b. An intervertebral disc 32 is disposed between the aligning vertebral bodies to act as a shock absorber and joint. Also as shown in FIGS. 2A and 2B, when adjacent vertebrae are aligned, the inferior articular processes 28 of the superior vertebra 22a are adjacent to the superior articular processes 25 of the inferior vertebra 22b. The adjacent vertebrae are movable (i.e., to allow for bending and twisting) relative to each other via facet joints 34 formed on articulating faces of the articular processes. Two facet joints 34, each defined by superior and inferior facet components, are formed between the adjacent superior and inferior vertebrae 22a, 22b.

The facet joints 34 are in almost constant motion with the spine, and degeneration of the joints is not uncommon. Such degeneration alters the mechanics of the facet joints and may cause spinal stenosis, degenerative spondylolisthesis, and degenerative scoliosis. Moreover, the posterior spinal arch is known to degenerate due to congenital defects and/or tissue degeneration.

These and other maladies are often treated with spinal arthrodesis, i.e., spine fusion, which is a surgical procedure in which the adjacent vertebrae are fused after removal of degenerated tissue and bone. Some joint replacement methods also are known to treat the condition. However, while spinal arthrodesis may alleviate the immediate symptoms, all motion between the vertebrae that are fused is lost. Moreover, the known joint replacements tend to be overly complex and cumbersome.

Accordingly, there is a need in the art for an improved spinal arthroplasty that avoids the drawbacks and disadvantages of known surgical techniques and implants.

### SUMMARY OF THE INVENTION

This invention addresses the foregoing needs in the art by providing a prosthesis and a method for replacing one or both of degenerated facet joints and a degenerated posterior arch.

In one aspect of the present invention, a posterior arch prosthesis includes a main body, transverse protuberances extending generally outwardly from the main body, articulating surfaces formed on each of the transverse protuberances and a posterior protuberance extending generally posteriorly from the main body portion. The prosthesis is attached to a first vertebra from which the natural posterior arch has been removed such that the articulating surfaces are arranged in articulating contact with surfaces on an adjacent, second vertebra. The posterior protuberance includes an attachment to which native structure may be reattached upon attachment of the prosthesis to the first vertebra.

In another aspect of the invention, a system for replacing a posterior arch and facet joints between adjacent vertebrae includes a first prosthesis and a second prosthesis. The first prosthesis is secured to one of a superior vertebra and an inferior vertebra and has at least one substantially concave surface. The second prosthesis is secured to the other of the superior vertebra and the inferior vertebra, and has at least one substantially convex surface. The first prosthesis and the second prosthesis, when secured to the respective vertebrae, are disposed such that the substantially concave surface of the first prosthesis and the substantially convex surface of the second prosthesis are proximate to each other, to allow for articulation of the superior vertebra with respect to the inferior vertebra. At least one of the first prosthesis and the second prosthesis replaces a posterior arch of the vertebra to which the prosthesis is secured.

In a still further aspect of the invention, a method of replacing degenerated sections of adjacent vertebrae includes removing a portion of a process having a degenerated facet and the posterior arch from a first of the adjacent vertebrae and removing a portion of a process having a degenerated facet from the second of the adjacent vertebrae. The method also includes securing a first prosthesis to the first of the adjacent vertebrae and securing a second prosthesis to the second of the adjacent vertebrae. The first prosthesis has a generally concave portion and a shape to approximate the function of the removed posterior arch. The second prosthesis has a generally convex portion. The first prosthesis and the second prosthesis are secured to the respective vertebrae such that the generally concave portion of the first prosthesis and the generally convex portion of the second prosthesis are adjacent each other, to articulate the adjacent vertebrae.

A better understanding of these and other aspects, features, and advantages of the invention may be had by reference to the drawings and to the accompanying description, in which preferred embodiments of the invention are illustrated and described.

### BRIEF DESCRIPTION OF THE DRAWINGS

FIG. 1 is a side elevation schematic view of the lumbar spinal region and the sacrum region in a healthy human.

FIG. 2A is a detailed side perspective view showing two adjacent lumbar vertebrae, as found in a healthy human.

FIG. 2B is a detailed posterior perspective view showing two adjacent lumbar vertebrae, such as those shown in FIG. 2A.

FIG. 2C is a top plan view of a vertebrae.

FIGS. 3A and 3B, respectively, are a perspective view and a side view of an arch prosthesis according to a preferred embodiment of the invention.

FIG. 4 is a perspective view of a portion of a spinal column illustrating an application of the arch prosthesis according to the preferred embodiment.

FIG. 5 is a posterior perspective view of the portion of the spinal column with applied prosthesis of FIG. 4.

FIG. 6 is a side perspective view of the portion of the spinal column with applied prosthesis of FIG. 4.

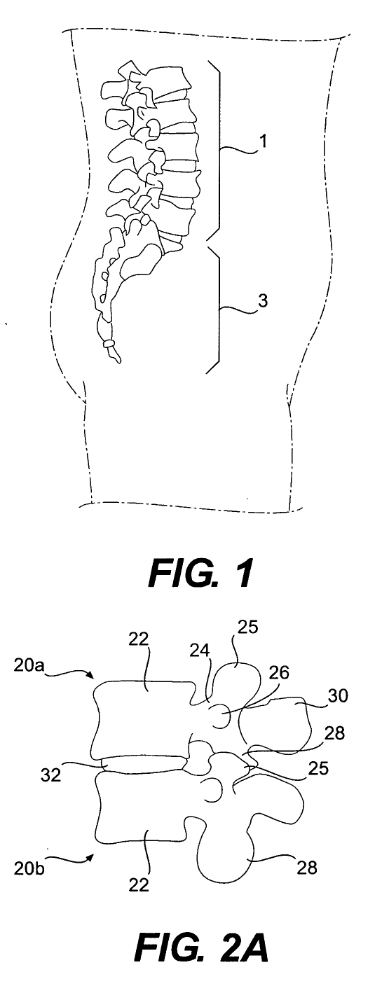

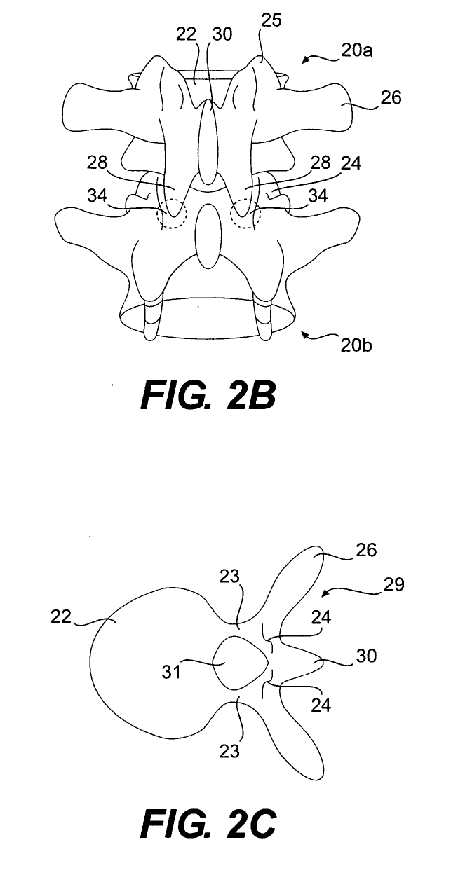

Throughout the figures, like or corresponding reference numerals have been used for like or corresponding parts.

### DETAILED DESCRIPTION OF THE PREFERRED EMBODIMENTS

The drawings illustrate embodiments of a prosthesis for replacing one or both of a posterior arch and an intervertebral facet joint. Various embodiments of the prosthesis according to the principles of the disclosure may be used for treating any problems that lend themselves to joint and/or degenerative bone and tissue replacement.

Throughout the application, terms such as posterior, anterior, superior, inferior, rostral, and caudal are used in some embodiments to describe positions of components of the embodiments. While the meanings of such terms are generally used in the art to describe certain positions, such are used herein merely as relative positions and/or modifiers to describe relative positions. Components labeled using such terms in an illustrated embodiment are not intended to limit the scope of claims to any device or method.

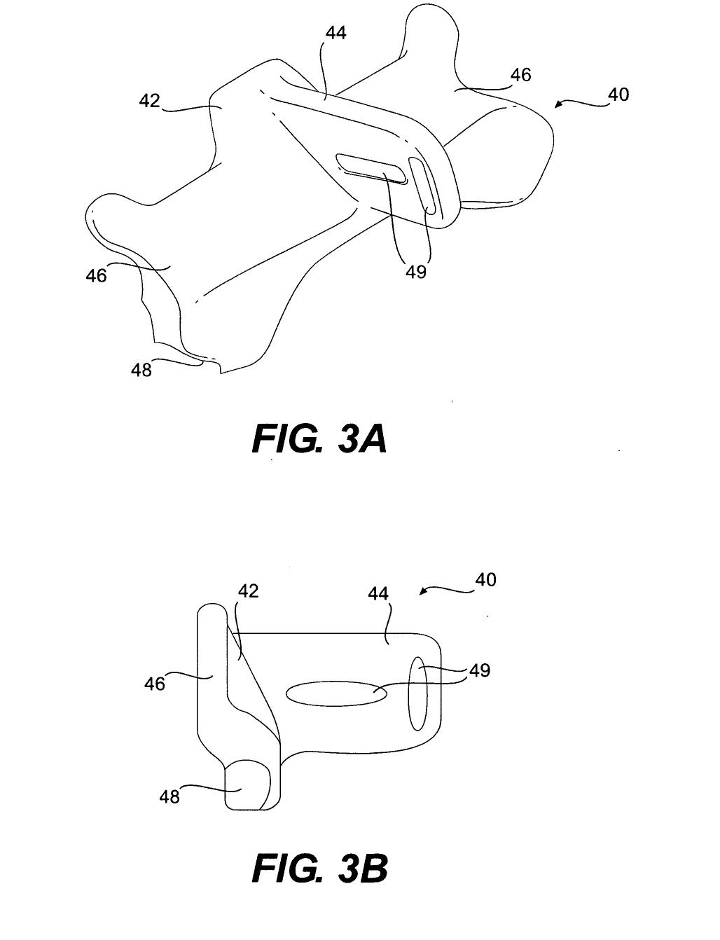

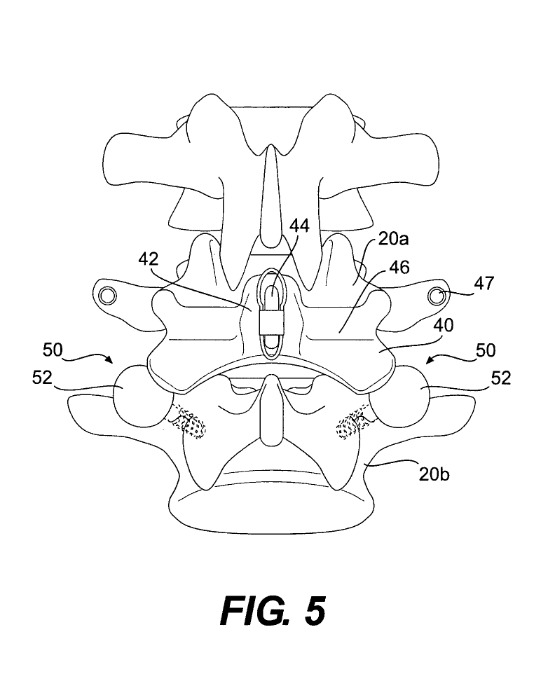

A preferred embodiment of the present invention will be described with reference to FIGS. 3A, 3B, and 4-6.

FIGS. 3A and 3B depict a prosthesis 40 according to a preferred embodiment of the invention. The prosthesis generally includes a main body portion 42, a posterior protuberance 44 extending generally posteriorly from the main body portion 42, and two transverse protuberances 46 extending generally laterally outwardly from the main body portion 42. Preferably, a generally concave indentation, or cupped portion, 48 is formed on an outer or distal surface of each of the transverse protuberances 46. The function of each of these features will be described in more detail below, in conjunction with a discussion of the intended use of the prosthesis 40.

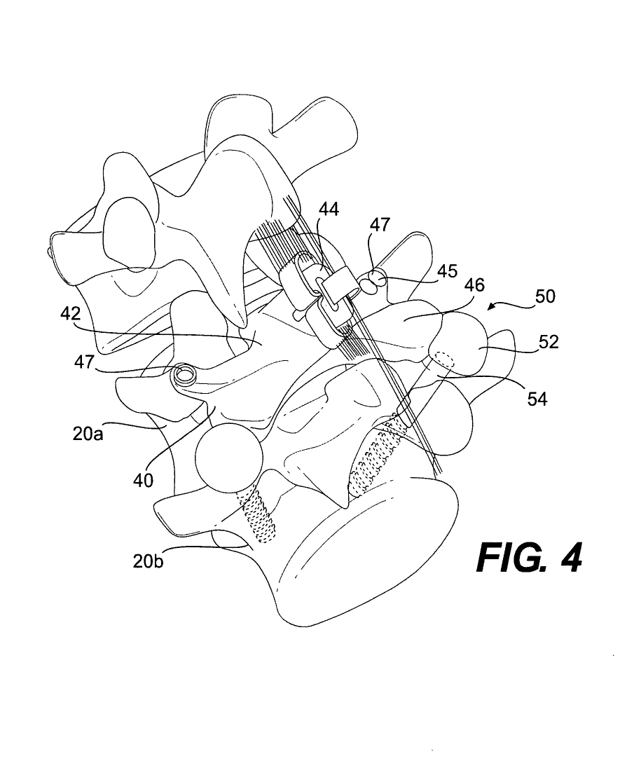

FIGS. 4-6 illustrate the prosthesis 40 as used to replace an ineffective facet joint between a superior vertebra 20a and an inferior vertebra 20b and a degenerated posterior arch 29 on the superior vertebra 20a. In the preferred embodiment, the prosthesis 40 is attached (discussed later) to the superior vertebra 20a such that the indentations 48 are arranged with the concavity opening generally in a caudal direction. The indentations 48 preferably also are arranged with the concavity slightly outwardly and anteriorly facing. This concavity forms one half of a new joint between the superior and inferior vertebrae 20a, 20b. The other half of the joint, i.e., the portion of the inferior vertebra 20b with which the prosthesis 40 articulates, is comprised of two inferior prostheses 50 secured (also discussed later) to the inferior vertebra 20b. As illustrated, each of the two inferior prostheses 50 includes a domed portion 52 having a generally convex shape. When the prosthesis 40 is attached to the superior vertebra 20a, and the inferior prostheses 50 are attached to the inferior vertebra 20b, the concavity of the indentations 48 of the prosthesis 40 articulates with the convexity of the domed portions 52 of the inferior prostheses 50, thereby forming a joint between the superior and inferior vertebrae 20a, 20b.

The preferred prosthesis 40 of this embodiment also replaces the posterior arch, i.e., the laminae 24 and any other structure bridging the two pedicles 23. Specifically, as will be described in more detail below, some or all of the degenerative arch is preferably removed prior to attachment of the prosthesis 40. When attached, the main body portion 42 and the transverse protuberances 46 bridge the gap between the pedicles 23, thereby recreating the posterior arch.

In a natural facet joint, the structure of each facing surface allows articulation therebetween, but also restricts motion to some extent, e.g., to disallow movements that would be harmful to the spinal column. Similarly, the present invention is designed to allow for a limited range of relative motion. Specifically, restricted movement is allowed between the concave indentations 48 on the prosthesis 40 and the convex domed portion 52 of the inferior prostheses 50. This restriction of the movement may be achieved through known design techniques. For example, instead of making the concave and convex surfaces completely complementary, one or both surfaces may include surface irregularities, in the form of flatter or more arcuate contours. By making a radius of curvature of the concave portion greater than that of the convex portion, for example, more movement will be allowed. Meanwhile, flatter areas in the concave portions may act to impinge relative movement between the components. Adjusting the direction in which the concavity opens may also dictate the relative motion of the superior and inferior vertebrae. Thus, according to the preferred embodiment of the invention, the curvatures of the concave and convex portions may be formed to allow for articulation nearly identical to a natural facet joint, or more or less articulation may be achieved, as desired.

For ease of use, in the preferred embodiment, any irregularities formed to facilitate/restrict articulation of the vertebrae 20a, 20b are preferably contained on the concave indentation of the prosthesis 40, while the domed portion 52 of the inferior prostheses 50 is preferably substantially hemispherical, with no irregularities. In this manner, the inferior prostheses 50, which preferably have a threaded portion 54 upon which the domed portion 52 is mounted, may be screwed into the inferior vertebra 20b to a desired torque, without concern for the rotational position of the prostheses 50. Of course, the domed portions 52 may have irregularities as described above, but such an arrangement would likely require that a skilled surgeon properly align the inferior prostheses 50 upon attachment.

As generally described to this point, the preferred embodiment includes a prosthesis 40 attached to a superior vertebra 20a for articulation with dome-shaped prostheses 50 attached to an inferior vertebra 20b. Specifically, the generally concave indentations 48 of the prosthesis 40, which effectively replace the inferior articular facets of the superior vertebra 20a, articulate with the generally convex heads 52 of the inferior prostheses 50, which effectively replace the superior articular facets processes of the inferior vertebra 20b. This arrangement, however, is in contrast to natural human anatomy, in which the facets formed on the inferior articular processes are generally convex in shape, for articulation with generally concave facets of the superior articular processes. The preferred arrangement disclosed in this embodiment is believed to be beneficial, for example, in preventing forward slip between the vertebrae. Of course, our invention also contemplates the convex portion on the superior vertebra 20a and the concave portion on the inferior vertebra 20b. For example, such may be accomplished by attaching the prothesis 40 to the inferior vertebra 20b and the inferior prostheses 50 to the superior vertebra 20a, or by forming convex surfaces on the prosthesis 40 and articulating concave surfaces on the inferior prostheses 50.

As is generally understood in the art, before the components of the prosthetic system are attached to the vertebrae, it may be necessary and/or desirable to prepare the vertebrae. Specifically, any and all degenerated bone and tissue may be removed and the area to which the prosthesis 40 and/or inferior prostheses 50 are to be attached may be altered to better accommodate the prostheses. For example, as illustrated in the figures and discussed above, the present embodiment is suited for replacement of the posterior arch, so all of the laminae 24 may be removed from the superior vertebra 20a. Removal of the arch also entails removal of at least the spinous process 30. The vertebrae may also further be altered, for example, by removal or reduction of one or more of the natural articular processes or any other process and/or native structure.

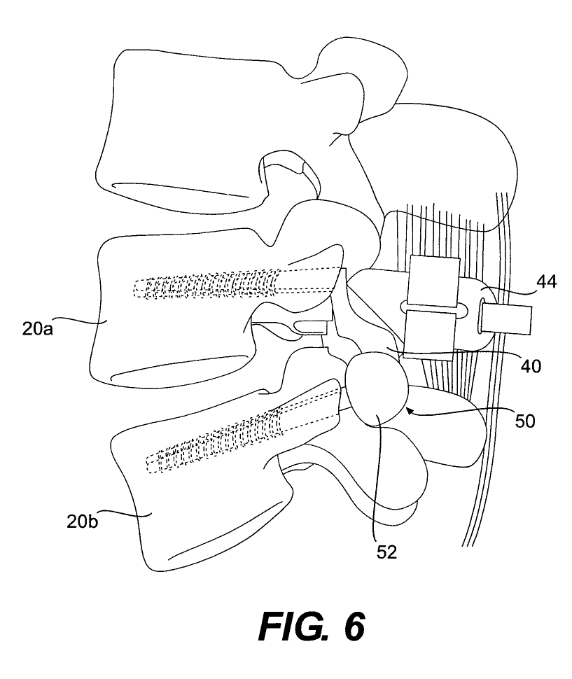

Any alteration of the vertebrae may pose additional problems. For example, when the posterior arch, and thus the spinous process 30, is removed from the superior vertebra 20a to allow for attachment of the prosthesis 40, any ligaments or other native structure that were attached to the spinous process 30 must be reattached in some manner. For this reason, the prosthesis 40 includes the posterior protuberance 44, extending posteriorly from the main body 42. With the prosthesis 40 attached to the vertebra, the posterior protuberance 44 may serve as an attachment point to which native structure is reattached, as specifically shown in FIG. 6. Specifically, the posterior protuberance 44 of the preferred embodiment may include one or more apertures 49, as shown in FIGS. 3A and 3B, to which native structure can be sutured, or otherwise attached. Alternatively, or in addition, the posterior protuberance 44 may be covered in a known substance to which the native structure may be attached. For example, the protuberance may be covered with a compliant, sew-able material, thus providing a surface to which ligaments and tendons can be attached. Such material may be, for example, a sewing ring as used on a mechanical heart valve, a hydrogel (which may be cell-seeded or growth factor-seeded or otherwise doped), or a similar material. Preferably, the material provides a layer through which a suture needle may simply be passed. The material may also be provided on other surfaces of the protrusion 40, as it is contemplated that reattachment of native structure may be done at a portion of the prosthesis 40 other than the posterior protuberance 44.

The prosthesis 40 is preferably attached to the superior vertebra via screws 45 through holes 47 formed in the transverse protuberances 46 of the prosthesis 40. The screws 45 preferably are threaded into the exposed pedicles 23 (i.e., after removal of the laminae 24) of the vertebra, although such is not required. The screws may be inserted into any portion of the vertebra or surrounding structure. Moreover, the invention is not limited to the use of screws as a fastener for securing the prosthesis 40 to the vertebra. For example, any mechanical or equivalent means that secures the prosthesis relative to the vertebra may be used, including, but not limited to, clamps, bolts, forks, spikes, latches, or adhesives, including polymethylmethacrylate. As noted above, the inferior prostheses 50 preferably include a domed head 52 formed on a threaded body 54. In use, the threaded body 54 is screwed directly into the inferior vertebra 20b, leaving only the domed head 52 extending from the vertebra. The threaded body 54 is preferably screwed into the pedicle 23 of the inferior vertebra 20b, although it may alternately be threaded into any portion of the vertebra or surrounding structure. Any arrangement in which the domed portion 52 may articulate with the indentations 48 of the prosthesis 40 on the superior vertebra 20a is believed to be within the scope of the invention. The inferior prostheses may also be attached by some other fastener, or known mechanical or equivalent means, including but not limited to, clamps, bolts, forks, spikes, latches, or adhesives, including, but not limited to, polymethylmethacrylate. As needed, the prosthesis 40, inferior prostheses 50, and attachment means may also be coated with a beaded material or plasma spray to promote bony ingrowth and a firm connection therebetween.

The prosthesis 40 and the inferior prostheses 50 may be formed of a plastic such as high molecular weight polyethylene or PEEK, of a ceramic material, of a metal, such as stainless steel, titanium, cobalt alloy, of natural or synthetics fibers, such as carbon fiber or rubber, or of any other suitable materials. In addition, each of the components may be made up of more than one material. For example, the body of the prosthesis may be made of one material, while the point of attachment to the pedicles may be beaded or made of roughened titanium or hydroxylapatite, to provide an area with a high affinity for bone ongrowth or ingrowth. Moreover, because a feature of the invention is an articulating joint between the vertebrae, relative movement between the concave indentations of the prosthesis 40 and the convex dome of the inferior prostheses 50 are preferably aided by polishing or coating the surfaces of the indentation and dome. For example, if the surfaces are made of metal, the metal surfaces may be polished metal.

While it should be readily apparent to a skilled artisan from the discussion above, a brief description of a suitable surgical procedure that may be used to implant the prosthetic system according to the invention will now be described. Generally, the artificial joint may be implanted into a body using a posterior transforaminal approach. According to this approach, an incision, such as a midline incision, is made in a patient's back. The vertebrae may then be prepared for attachment of the prostheses. As discussed above, this preparation may include, among other things, removing bone and tissue constituting the posterior arch, removing one or more process from the adjacent vertebrae, and/or trimming the existing facet joints. The prosthesis and inferior prostheses are then inserted through the incision, and attached to the vertebrae, by means described above.

Although a preferred embodiment of the invention has been described, several variations and alternative embodiments also are contemplated.

As an example, in one alternate embodiment of the invention, the inferior prostheses are not needed. Particularly, it is envisioned that only the facets and/or posterior arch need be replaced on one of the superior and inferior vertebrae. The prosthesis in this instance similar to the prosthesis 40 discussed above in the preferred embodiment, but the indentations 48 will be formed to articulate with the healthy facets on the adjacent, healthy vertebra. Of course, the indentations may, in fact, not be indentations at 11, but convex in shape to articulate with a generally concave facet. In this manner, a simple, singular device is provided that replaces a degenerated arch, and maintains relative movement of the affected vertebra relative to the adjacent vertebra.

Additionally, while the inferior prostheses 50 have been discussed above as separate prostheses, in another embodiment of the invention a single prosthesis may include the two domed portions 52. As will be understood, a single prosthesis may require further preparation of the inferior vertebra prior to attachment thereto. For instance, it may be necessary to remove all or a portion of the spinous process 30 of the inferior vertebra to accommodate the singular prosthesis. Removal of the spinous process 30 would also require detachment of native structure that normally would be attached to the spinous process, and an alternate structure, such as the posterior protrusion of the prosthesis 40 discussed above, may then be required for re-attachment of that native structure. Similarly, the singular prosthesis could also function as a prosthetic arch, in much the same manner as the prosthesis 40 of the preferred embodiment. Alternatively, the singular prosthesis could cooperate with all of the features of the natural vertebra, thereby minimizing, and potentially eliminating, removal of any bone and tissue from the vertebra.

Similar to the embodiment just described, it is also envisioned that the prosthesis 40 could consist of more than one component. In particular, the prosthesis may include first and second components, one for attachment to each of the left and right pedicles, similar to the inferior prostheses. Each of the components would preferably include a generally concave indentation for articulation with one of the inferior prostheses. This embodiment may be particularly beneficial if the arch need not be replaced.

It is also envisioned that the prosthesis 40 and/or inferior postheses according to the invention allow for vascularization of either or both of the surface and the interior of the device. For example, when native structure is reattached to the prostheses, that native structure may not readily receive an appropriate nutritional supply. Thus, to promote supply of needed nutrition, the interior of the prosthesis could be open celled. Alternatively, the interior of the prosthesis could be made with pre-created tubes or channels through which blood is intended to pass. In this manner, a vessel could be connected only to either end of the device to supply and drain the device. In other embodiments, the interior of the prosthesis may be seeded with materials that encourage angiogenesis, or could contain bioactive agents that may be eluted from the device.

The embodiments discussed above are representative of embodiments of the present invention and are provided for illustrative purposes only. They are not intended to limit the scope of the invention. Variations and modifications are apparent from a reading of the preceding description and are included within the scope of the invention. The invention is intended to be limited only by the scope of the accompanying claims.

What is claimed is:

1. A posterior arch prosthesis, comprising:
a main body;
transverse protuberances extending generally outwardly from said main body;
articulating surfaces formed on each of said transverse protuberances; and
a posterior protuberance extending generally posteriorly from said main body,
wherein said transverse protuberances are attached to a first vertebra from which a natural posterior arch has been removed such that said articulating surfaces are arranged in articulating contact with surfaces on an adjacent, second vertebra, and
wherein said posterior protuberance includes an attachment portion to which native structure may be reattached upon attachment of the prosthesis to the first vertebra.

2. A posterior arch prosthesis according to claim 1, further comprising a fastener that attaches said transverse protuberances to pedicles of the first vertebra.

3. A posterior arch prosthesis according to claim 1, wherein said transverse protuberances are attached to the first vertebra such that the articulating surfaces are arranged to articulate with surfaces of one or more prostheses attached to the second vertebra.

4. A posterior arch prosthesis according to claim 1, wherein said transverse protuberances are attached to the first vertebra such that the articulating surfaces are arranged to articulate with existing facets of the second vertebra.

5. A posterior arch prosthesis according to claim 1, wherein said attachment portion includes one or more of apertures formed through said posterior protuberance and a sew-able material.

6. A posterior arch prosthesis according to claim 1, wherein said prosthesis is open-celled to promote vascularization.

7. A posterior arch prosthesis according to claim 1, further comprising internal channels to promote vascularization.

8. A posterior arch prosthesis according to claim 6, wherein said prosthesis is seeded with materials that promote angiogenesis.

9. A system for replacing facet joints between adjacent vertebrae, the system comprising:
a first prosthesis secured to one of a superior vertebra and an inferior vertebra, said first prosthesis having at least one substantially concave surface; and
a second prosthesis secured to the other of the superior vertebra and the inferior vertebra, said second prosthesis having at least one substantially convex surface,
wherein said first prosthesis and said second prosthesis, when secured to the respective vertebrae, are disposed such that the substantially concave surface of said first prosthesis and the substantially convex surface of said second prosthesis are proximate to each other, to allow for articulation of the superior vertebra with respect to the inferior vertebra, and
wherein at least one of said first prosthesis and said second prosthesis replaces a posterior arch of the vertebra to which the prosthesis is secured.

10. A system according to claim 9, wherein at least one of said first prosthesis and said second prosthesis is a unitary structure.

11. A system according to claim 9, wherein at least one of said first prosthesis and said second prosthesis comprises more than one component.

12. A system according to claim 9, wherein said second prosthesis comprises two prostheses, each including a dome-shaped head disposed on a threaded portion, said threaded portion being inserted into the inferior vertebra in use.

13. A system according to claim 9, wherein one of the convex surface of said second prosthesis and the concave surface of said first prosthesis includes surface irregularities, such that articulation of the concave surface with respect to the convex surface is limited.

14. A system according to claim 9, wherein at least one of said first prosthesis and said second prosthesis includes attachment means to which native structure is attached.

15. A system according to claim 14, wherein said attachment means includes at least one of a protuberance extending from said first prosthesis and a sew-able material.

16. A system according to claim 9, wherein the concavity of said first prosthesis opens generally outwardly and rostrally, for articulation with the convex surface of said second prosthesis.

17. A system according to claim 9, wherein at least one of the first prosthesis and the second prosthesis includes one or more internal channels, to promote vascularization.

18. A system according to claim 17, wherein said first prosthesis and said second prosthesis have an open celled structure, to promote angiogenesis.

19. A method of replacing degenerated sections of first and second adjacent vertebrae, the method comprising the steps of:
removing a process having a degenerated facet and the posterior arch from the first vertebrae;
removing a process having a degenerated facet from the second vertebrae;
securing a first prosthesis to the first vertebrae, the first prosthesis having a generally concave portion and having a shape to approximate the function of the removed posterior arch; and
securing a second prosthesis to the second vertebrae, the second prosthesis having a generally convex portion,
wherein the first prosthesis and the second prosthesis are secured to the respective vertebrae such that the generally concave portion of the first prosthesis and the generally convex portion of the second prosthesis are adjacent each other, to articulate the first and second adjacent vertebrae.

20. A prosthetic system that replaces a degenerated coupling between articulated facets of adjacent vertebrae, the prosthetic system comprising:
a first prosthetic article for securement to a superior vertebra, said first prosthetic article having a first surface; and
a second prosthetic article for securement to an inferior vertebra adjoining the superior vertebra, said second prosthetic article having a second surface,
wherein when said first prosthetic article and said second prosthetic article are secured to the respective vertebrae, the first surface and the second surface are arranged proximate to each other, so as to articulate said first prosthetic article and said second prosthetic article, thereby articulating the superior vertebra and the inferior vertebra.
